(19) United States
(12) Patent Application Publication
(10) Pub. No.: US 2026/0242321 A1
(43) Pub. Date: Aug. 20, 2026

(54) PROCESS FOR PREPARING, BY HYDROFORMYLATION, SHORT-CHAIN OLEFINS IN THE GAS PHASE

(71) Applicant: Evonik Oxeno GmbH & Co. KG, Marl (DE)

(72) Inventors: Robert Franke, Marl (DE); Linda Arsenjuk, Dortmund (DE); Corina Nentwich, Datteln (DE); Frank Stenger, Alzenau (DE); Marc Oliver Kristen, Haltern am See (DE); Peter Kreis, Dortmund (DE)

(73) Assignee: Evonik Oxeno GmbH & Co. KG, Marl (DE)

(21) Appl. No.: 18/995,310

(22) PCT Filed: Apr. 26, 2023
(86) PCT No.: PCT/EP2023/060930

§ 371 Date: Jan. 16, 2025

(30) Foreign Application Priority Data
Jul. 19, 2022 (EP) 22185749.3

(51) Int. Cl. C07C 45/50; B01J 27/224; B01J 31/16; B01J 35/56

(52) U.S. Cl. CPC C07C 45/50; B01J 27/224; B01J 31/1616; B01J 35/56; B01J 2231/321; B01J 2531/822

(57) ABSTRACT

A process for hydroformylates short-chain olefins in a reactor in which the catalyst system is present in heterogenized form on a support composed of a porous ceramic material and wherein synthesis gas or carbon monoxide is passed through the reactor during process idle times.

The project that resulted in this patent application was supported under grant No. 869896 of the European Union's Horizon 2020 research and innovation program.

The present invention relates to a process for hydroformylating short-chain olefins, especially C2 to C5 olefins, in a reactor in which the catalyst system is present in heterogenized form on a support composed of a porous ceramic material and wherein synthesis gas or carbon monoxide is passed through the reactor during process idle times.

Hydroformylation is one of the most important reactions in industrial chemistry, having an annual global production capacity of several million tonnes. It involves reacting alkenes (olefins) with a mixture of carbon monoxide and hydrogen (also: synthesis gas or syngas) using a catalyst to give aldehydes, which are important and valuable intermediates in the production of chemical bulk products such as alcohols, esters or plasticizers.

Hydroformylation is on the industrial scale carried out exclusively under homogeneous catalysis. The soluble transition metal catalyst systems are typically based on cobalt or rhodium, which is often used together with phosphorus-containing ligands, for example phosphines or phosphites, for the hydroformylation of comparatively short-chain olefins.

There are various problems with the known processes, these problems being linked in particular to the fact that both rhodium and cobalt and compounds thereof are comparatively costly. Large amounts of energy and outlay on process are necessary in order for catalyst losses during the hydroformylation process to be avoided as far as possible, for example through catalyst recycling steps, some of which are very laborious. Moreover, product purification steps are becoming more laborious in order to ensure that as far as possible no catalyst residues remain in the product.

Further problems with the known homogeneously catalysed processes are the stability of the ligands, which have to withstand the conditions of the hydroformylation, such as temperature, pressure, pH etc., and the consumption during the process of the solvent used, which has to be compensated for by replenishment.

In order to get around the abovementioned problems in homogeneously catalysed hydroformylation, hydroformylation processes have been developed in which the catalyst is heterogenized, especially by immobilization on a support material. The terms “heterogenization” and “immobilization” should accordingly be understood as meaning that the catalyst is immobilized through the formation of a thin liquid film with the aid of an ionic liquid on the surface and/or in the pores of a solid support material and that there is no reaction solution in the conventional sense in which the catalyst is homogeneously dissolved. Hydroformylation processes in which the catalyst is present on a support material in a heterogenized form are disclosed for example in WO 2015/028284 A1, EP 3 632 885 A1, EP 3 744 707 A1, EP 3 632 886 A1 or in EP 3 736 258 A1.

When carrying out hydroformylation on heterogenized catalysts, problems can arise when idle times occur, for example during maintenance work or for other production-related reasons. Idle time is in the context of the present invention understood as meaning that during this time it is not possible to pass any feed mixture through the reactor and that it is accordingly also not possible for any hydroformylation to take place. After such an idle time, the reaction usually shows poorer performance, i.e. lower conversions and selectivities. This is presumably due to the formation of high boilers and the destruction of some of the catalyst. In order to avoid the formation of high boilers, during idle times the product mixture is normally purged from the reaction space with nitrogen. This was nevertheless found to be followed by a decrease in reactor performance.

The object of the present invention was therefore to provide a process for hydroformylating olefins that does not have the abovementioned problems and that is in particular able, after an idle time, to be restarted more quickly and without loss of catalyst activity.

This object is achieved according to claim 1 in that the reactor is purged during the idle time with synthesis gas or with carbon monoxide. Product residues are accordingly purged from the reaction space and the catalyst complex is protected by the high CO partial pressure.

The present invention thus provides a process for hydroformylating C2 to C8 olefins in a reaction zone using a heterogenized catalyst system, wherein a gaseous feed mixture containing the C2 to C8 olefins is passed together with synthesis gas in at least one reactor over a support arranged in the at least one reactor that is composed of a porous ceramic material on which the catalyst system, which comprises a metal from group 8 or group 9 of the periodic table of the elements, at least one organic phosphorus-containing ligand, a stabilizer and optionally an ionic liquid, is present in a heterogenized form; wherein the support is a monolith, i.e. a block of a ceramic material, or is present in the form of a powder, in the form of a granular material or in the form of shaped bodies and the support consists of a carbidic, nitridic, silicidic material or mixtures thereof, characterized in that
idle times occur during the process in which no gaseous feed mixture is passed through the reactor, the reactor being purged during the idle time with synthesis gas or carbon monoxide.

The characterizing feature of the present invention is the purging of the reactor with synthesis gas and carbon monoxide in the idle time. After the idle time this makes it possible, compared to other purge gases, to achieve higher conversions and yields and for the reactor to accordingly return more swiftly to normal operation. The temperature during the purging with synthesis gas or carbon monoxide is preferably in the range from 20 to 200° C., further preferably in the range from 22 to 175° C. and more preferably in the range from 85 to 150° C. It is additionally preferable that the reactor is during the idle time maintained at a maximum of less than 10° C. below the reaction temperature. Cooling should thereby be avoided. The pressure during purging is relatively noncritical, but should not exceed the pressure during the hydroformylation.

The feed mixture used may be any mixture comprising C2 to C8 olefins, preferably C2 to C5 olefins, especially ethene, propene, 1-butene, 2-butene, 1-pentene or 2-pentene, as reactants. The amount of olefins in the feed mixtures should understandably be high enough to be able to economically operate a hydroformylation reaction. The feed mixtures that may be employed in the process according to the invention include in particular also technical mixtures from the petrochemical industry, for example raffinate streams (raffinate I, II or III) or crude butane. According to the present invention, crude butane comprises 5% to 40% by weight of butenes, preferably 20% to 40% by weight of butenes (the butenes are composed of 1% to 20% by weight of 1-butene and 80% to 99% by weight of 2-butene), and 60% to 95% by weight of butanes, preferably 60% to 80% by weight of butanes.

The process according to the invention is carried out in at least one reactor in which the hydroformylation according to the invention takes place. The support with the heterogenized catalyst system is arranged in the at least one reactor. In a further embodiment of the present invention, the process can also be carried out in a plurality of reactors, which may be connected in parallel or in series. Preferably, the reactors are in the present case connected in parallel and are used alternately.

The hydroformylation is preferably carried out under the following conditions. The temperature in the hydroformylation should be in the range from 65 to 200° C., preferably 75 to 175° C. and more preferably 85 to 150° C. The temperature may be set by means of a suitable cooling apparatus, for example a cooling jacket. The pressure during the hydroformylation should not exceed 35 bar, preferably 30 bar, more preferably 25 bar. The molar ratio between synthesis gas and the feed mixture should be between 6:1 and 1:1, preferably between 5:1 and 3:1. Optionally, the feed mixture can be diluted with inert gas, for example with alkanes present in technical hydrocarbon streams.

The catalyst system used in the hydroformylation process according to the invention preferably comprises a transition metal from group 8 or 9 of the periodic table of the elements, especially iron, ruthenium, iridium, cobalt or rhodium, further preferably cobalt and rhodium, more preferably rhodium, at least one organic phosphorus-containing ligand and a stabilizer.

The stabilizer is preferably an organic amine compound, more preferably an organic amine compound containing at least one 2,2,6,6-tetramethylpiperidine unit of formula (I):

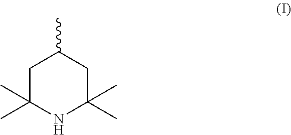

In a particularly preferred embodiment of the present invention, the stabilizer is selected from the group consisting of the compounds of the following formulas (I.1), (I.2), (I.3), (I.4), (I.5), (I.6), (I.7) and (I.8).

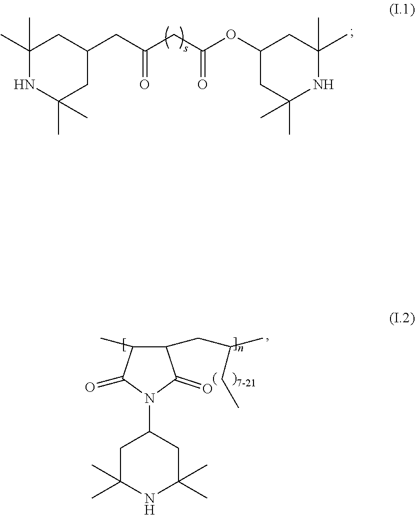

where n is an integer from 1 to 20;

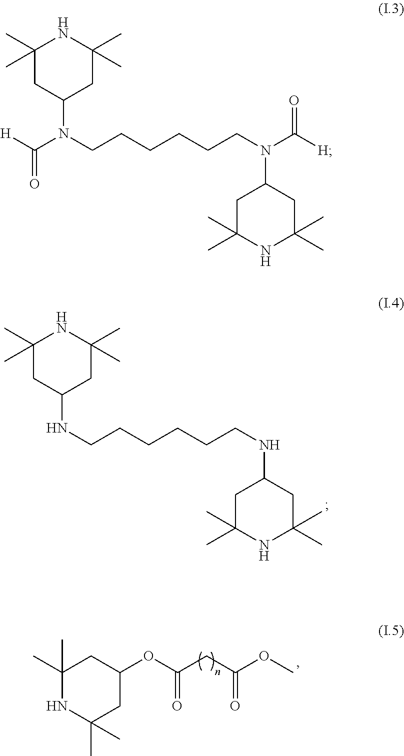

where n is an integer from 1 to 12;

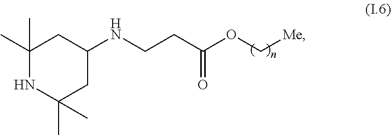

where n is an integer from 1 to 17;

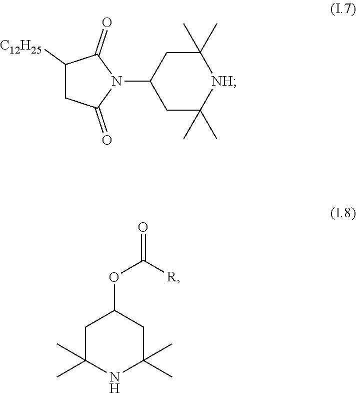

where R is a C6- to C20-alkyl group.

For all film-forming components, i.e. in this case the stabilizer, the gas solubility for the reactants should be better than the gas solubility of the products. In that way alone, it is possible to achieve partial physical separation between reactant olefins used and product aldehydes formed. In principle, other film-forming substances would also be conceivable for the purpose, but care should be taken to ensure there is no increased formation of high boilers and/or that the resupply of the reactant olefins is limited.

The organic phosphorus-containing ligand selected for the catalyst system according to the invention may be any ligand known for the hydroformylation. A large number of suitable ligands is known from the patent and specialist literature to those skilled in the art, for example mono- or biphosphite ligands. The organic phosphorus-containing ligand preferably has a biphosphite structure according to the general formula (II)

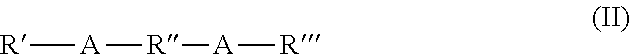

where R′, R″ and R″ are each organic radicals and the two A are each a bridging —O—P(—O)₂— group, wherein two of the three oxygen atoms —O— are attached respectively to the radical R′ and to the radical R′″, with the proviso that R′ and R′″ are nonidentical, The organic radicals R′, R″ and R′″ preferably do not contain any terminal trialkoxysilane groups.

In a preferred embodiment, R′, R″ and R′″ in the compound of the formula (VI) are preferably selected from substituted or unsubstituted 1,1′-biphenyl, 1,1′-binaphthyl and ortho-phenyl groups, especially from substituted or unsubstituted 1,1′-biphenyl groups, with the proviso that R′ and R″ are nonidentical. More preferably, the substituted 1,1′-biphenyl groups have an alkyl group and/or an alkoxy group in the 3,3′ and/or 5,5′ positions of the 1,1′-biphenyl base structure, especially a C1-C4 alkyl group, more preferably a tert-butyl and/or methyl group, and/or preferably a C1-C5 alkoxy group, more preferably a methoxy group.

The abovementioned catalyst system is according to the invention present in a heterogenized form on the support composed of a porous ceramic material. In the context of the present invention, the expression “in a heterogenized form on a support” is understood to mean that the catalyst system is immobilized on the inner and/or outer surface of the support through the formation of a thin, solid or liquid film with the aid of the stabilizer. The film may also be solid at room temperature and liquid under reaction conditions.

The inner surface of the solid support material comprises more particularly the inner surface area of the pores. The concept of immobilization includes both the situation where the catalyst system and/or the catalytically active species is present in dissolved form in the solid or liquid film and the situation where the stabilizer acts as an adhesion promoter or where the catalyst system is adsorbed on the surface, but not chemically/covalently bonded to the surface.

According to the invention, there is thus no reaction solution in the conventional sense in which the catalyst is homogeneously dissolved; instead, the catalyst system is dispersed on the surface and/or in the pores of the support.

The porous support material is preferably selected from the group consisting of a nitridic ceramic, a carbidic ceramic, a silicidic ceramic and mixtures thereof, for example carbonitridic materials.

The nitridic ceramic is preferably selected from silicon nitride, boron nitride, aluminium nitride and mixtures thereof. The carbidic ceramic is preferably selected from silicon carbide, boron carbide, tungsten carbide or mixtures thereof. Also conceivable are mixtures of carbidic and nitridic ceramic known as carbonitrides. The silicidic ceramic is preferably molybdenum silicide. The support according to the present invention to which the catalyst system is applied preferably consists of a carbidic ceramic, more preferably of silicon carbide.

The support may in the present case be present as a monolith, i.e. a block of a ceramic material, or in the form of a powder, in the form of a granular material or in the form of shaped bodies.

If the support is a monolith, the support consists of a block (a three-dimensional object) of the porous ceramic material. The block may either be in one-piece form or consist of multiple, i.e. at least two, individual parts that may be joined together to form the block and/or may be joined to one another in a fixed or partable manner.

The support composed of the porous ceramic material is preferably a component extending in three dimensions that may in principle be any geometric shape in its cross section, for example round, angular, square or the like. In a preferred embodiment, the component extending in three dimensions that can be used as support has a longitudinal direction (direction of the longest extent) in the main direction of through-flow (direction in which the feed mixture and the synthesis gas flow from the reactor inlet to the outlet).

The support monolith of the porous ceramic material thus shaped has at least one continuous channel in the main direction of through-flow. However, the channel(s) may also be configured such that they are not completely continuous but terminate at the opposite end from the reactor inlet, or the channel is closed towards this end. The support monolith may also have at least two or more channels. The diameter of the channels may be in the range from 0.25 to 50 mm, preferably in the range from 1 to 30 mm, further preferably in the range from 1.5 to 20 mm and more preferably in the range from 2 to 16 mm. If a plurality of channels is present, the diameters of the channels may be the same or different. The chosen diameter of the channels relative to the diameter(s) of the overall support should especially be such that the mechanical stability is unimpaired.

In addition, the support monolith of the ceramic material is porous, i.e. has pores. The catalyst system according to the invention is more particularly also present in the liquid or solid film in these pores. The pore diameter is preferably in the range from 0.9 nm to 30 μm, preferably in the range from 10 nm to 25 μm and more preferably in the range from 70 nm to 20 μm. Pore diameter can be determined by nitrogen adsorption or mercury porosimetry in accordance with DIN 66133 (1993-06 version).

In a preferred embodiment, the support monolith has at least partly continuous pores that extend from the surface to the channels and/or from one channel to the next channel(s). It is also possible that multiple pores are connected to one another and hence overall form a single continuous pore.

The support may according to the invention also be present in the form of a powder, in the form of a granular material or in the form of shaped bodies such as pellets, rings, spheres or the like. The average particle diameter (d50) of the support may be from 0.1 mm to 7 mm, preferably 0.3 to 6 mm, more preferably from 0.5 mm to 5 mm. The average particle diameter may be determined by imaging methods, in particular determined by the methods cited in the standards ISO 13322-1 (version: 2004 Dec. 1) and ISO 13322-2 (version: 2006 Nov. 1). The production of the support in the form of a powder, in the form of a granular material or in the form of shaped bodies can take place according to processes known to those skilled in the art. This may for example be done by mechanically comminuting a monolith of the carbidic, nitridic, silicidic material or mixtures thereof, for example using a jaw crusher, and adjusting the particle size of the resulting crushed granular material by sieving.

Like the support monolith, the particles of the powder, of the granular material or the shaped bodies composed of the ceramic material are porous, i.e. have pores. The catalyst system according to the invention is more particularly also present in the liquid or solid film in these pores. The pore diameter is preferably in the range from 0.9 nm to 30 μm, preferably in the range from 10 nm to 25 μm and more preferably in the range from 70 nm to 20 μm. The pore diameter can be determined by nitrogen adsorption or mercury porosimetry in accordance with DIN 66133 (1993-06 version).

Whether it be a monolith, powder, granular material or shaped bodies, the support is produced as described below:

To the provided pulverulent, granular or pellet-form support composed of the ceramic material, it is additionally possible to apply what is called a washcoat, which is based on the ceramic material of the support or is composed of the same or a different ceramic material, preferably silicon oxide. The washcoat itself may be porous or nonporous and is preferably nonporous. The particle size of the washcoat is preferably 5 nm to 3 μm, preferably 7 nm to 700 nm. The washcoat is used to introduce or to generate the desired pore size and/or to increase the surface area of the support. The washcoat can especially be applied by dipping (dip-coating) in a washcoat solution containing the ceramic material of the washcoat, optionally also in the form of a precursor. The amount of washcoat present on the support is ≤20% by weight, preferably ≤15% by weight, more preferably ≤10% by weight, based on the total amount of the support. However, in a preferred embodiment of the present invention the support does not have a washcoat.

The catalyst system is applied to the support with or without washcoat. For this purpose, a catalyst solution is first produced by mixing, especially at room temperature and ambient pressure, the catalyst solution comprising at least one organic phosphorus-containing ligand, at least one metal precursor, for example chlorides, oxides, carboxylates of the respective metal, at least one stabilizer and at least one solvent. It is optionally possible to use an ionic liquid in the production of the catalyst system, but the catalyst solution can also explicitly be prepared without ionic liquid. The catalyst solution should especially be produced in an inert environment, for example in a glovebox. “Inert environment” in this case means an atmosphere that is as far as possible free of water and oxygen.

The solvent may be chosen from all solvent classes (protic, aprotic, polar or nonpolar). A prerequisite for the solvent is the solubility of catalyst system (ligand, metal precursor, stabilizer and optionally the ionic liquid) and preferably also of the high boilers formed in the hydroformylation. Solubility can be increased during the immobilization step by heating.

The solvent is preferably aprotic and polar, for example acetonitrile and ethyl acetate, or else aprotic and nonpolar, for example THF and diethyl ether. It is also possible to use as solvent chlorinated hydrocarbons, for example dichloromethane, or aldehydes.

The catalyst solution thus produced is then contacted with the support (optionally including washcoat), for example by dipping (dip-coating) or by filling a pressure vessel, for example directly in the reactor (in-situ impregnation). If the catalyst solution is applied outside the reactor, the support must of course be reinstalled in the reactor after the solvent has been removed. Preferably, the catalyst solution is applied to the support with the washcoat directly in the reactor because this can avoid potentially time-consuming installation and deinstallation steps and potential contamination of the catalyst.

The reactor can be filled with the catalyst solution via the normal inlets/outlets, for example by means of a pump. Liquid distributors or nozzles within the reactor can ensure homogeneous distribution of the catalyst liquid, as can pressure drop internals or regulators for the metering rate that are optionally present.

After the catalyst system has been applied, the solvent is removed. This involves firstly discharging the residual catalyst solution via the reactor outlet. Solvent residues remaining in the reactor are then evaporated by adjusting the pressure or increasing the temperature. In another embodiment it is also possible for the pressure to be adjusted in tandem with an increase in temperature. The temperature may be 20 to 150° C., depending on the solvent. The pressure may be adjusted to a high vacuum (10⁻³ to 10⁻⁷ mbar), depending on the solvent, but overpressures of a few mbar up to several bar are also conceivable, depending on the solvent and temperature.

The stabilizer and the ionic liquid optionally present remain with the catalyst composed of the transition metal, especially cobalt or rhodium, and the organic phosphorus-containing ligand in heterogenized form on the support.

The catalyst system can be applied to the support either directly in the reactor (in situ) or outside the reactor. If the catalyst system is applied outside the reactor, the support must always be transported with exclusion of air, which can be achieved for example with a nitrogen countercurrent. In a preferred embodiment of the present invention, the catalyst system is applied directly in the reactor, i.e. in situ. After the solvent has been removed, the reactor can be used immediately and charged with the feed mixture. This has the advantage that no time-consuming installation and deinstallation steps that would result in a prolonged reactor shutdown are needed. Moreover, this then no longer gives rise to any limitation on the size of the support, in that suitable spaces having inert environments are available in a particular size. The size of the support can be chosen freely according to the reactor design.

Once the catalyst system has been applied to the support and the solvent has been removed, the plant, more particularly the reactor, can be started up, i.e. put into operation, through a two-stage or multistage startup procedure. A suitable startup procedure is described for example in EP 3 632 887.

A gaseous output comprising at least a portion of the product aldehydes formed and at least a portion of the unreacted olefins is preferably withdrawn continuously from the reaction zone in which the hydroformylation according to the invention is carried out. The gaseous output may be subjected to one or more physical separation step(s) in which the gaseous output is separated into at least one phase rich in unreacted olefins and at least one phase rich in product aldehyde.

The physical separation can be carried out by known physical separation methods such as condensation, distillation, centrifugation, nanofiltration or a combination of two or more thereof, preferably condensation or distillation.

In the case of a multistage physical separation, the phase rich in product aldehyde that is formed in the first physical separation is sent to a second physical separation, especially a downstream removal of aldehyde, in which the product aldehyde is separated from the other substances present in this phase, commonly alkanes and reactant olefins. The phase rich in unreacted olefin can be recycled to the hydroformylation step or, in the case of a multistage configuration, to one of the hydroformylation steps in order to further hydroformylate the olefins present therein to the product aldehyde.

In the physical separation, in addition to the phases mentioned it is also possible to withdraw a purge gas stream having a composition identical or at least similar to the phase rich in unreacted olefin. The purge gas stream can likewise be conveyed to the second physical separation or aldehyde removal in order to remove the product aldehydes present therein and to discharge impurities (for example nitrogen in the synthesis gas) or inert substances (for example alkanes in the feed mixture) from the system. The impurities or inert substances can typically be removed in the second physical separation as volatile substances, for example at the top of a column.

The present invention also further provides a plant with which the present process can be carried out and that especially comprises a reactor in which the hydroformylation step according to the invention is carried out. In addition, the plant may comprise a physical separation unit with which the gaseous output from the hydroformylation step is separated into at least one phase rich in unreacted olefin and at least one phase rich in product aldehyde, this physical separation unit being arranged downstream of the hydroformylation according to the invention. Present downstream of the first physical separation there may be a second physical separation unit, especially an aldehyde removal unit, with which the product aldehyde is removed.

Even without further elaboration it is assumed that those skilled in the art will be able to utilize the description above to the greatest possible extent. The preferred embodiments and examples are therefore to be interpreted merely as a descriptive disclosure which is by no means limiting in any way whatsoever.

The present invention is more particularly elucidated hereinbelow with reference to examples. Alternative embodiments of the present invention are obtainable by analogy therewith.

### EXAMPLE

### Experiments 1-4: Purging with Different Gases During System Idle Times

The starting material used for the support was SIC pellets (Sicat Sarl SIC-3). The SiC pellets were introduced into a round reactor sleeve 20 cm in length and having a diameter of one inch (approx. 2.54 cm), with glass beads of similar size introduced above and below the granular material. The SiC pellets were then loaded with a catalyst solution containing Rh(acac)(CO)₂, bisphephos (ligand), bis(2,2,6,6-tetramethyl-4-piperidyl)sebacate (stabilizer) and dichloromethane as solvent. This was done, after first purging the reactor with nitrogen, by introducing the catalyst solution into the reactor under a slight overpressure. After removal of the solvent from the reactor by discharge and evaporation, the catalyst system heterogenized on the granular support material was used for the hydroformylation.

The feed mixture used was a hydrocarbon stream having the following composition:

| | Amount |
|---|---|
| | (% by wt.) |
| Butenes | 13 |
| Butanes | 87 |

For the hydroformylation, the feed mixture was passed into the reactor together with synthesis gas (molar ratio of synthesis gas to feed mixture=3.5:1) at a total mass flow of 390 ml/min. The hydroformylation was carried out at a temperature of 120-130° C. and a pressure of 17 bar.

During the experiment, the flow of the reactants was paused for a number of relatively long periods (idle time) and various strategies for maintaining the activity of the catalyst system examined.

Experiment 1: In the first idle time 1, the reactor continued to be heated to 120° C. and was purged with a stream of nitrogen at a pressure of 1.2 bar.

Experiment 2: In the second idle time 2, the reactor continued to be heated to 120° C. and was purged with a stream of nitrogen at a pressure of 17 bar.

Experiment 3: In the third idle time 3, the reactor continued to be heated to 120° C. and was purged with synthesis gas at a pressure of 17 bar.

Experiment 4: In the fourth idle time 4, the reactor continued to be heated to 120° C. and was purged with synthesis gas at a pressure of 1.2 bar.

Before and after the respective experimental dates, the conversions and yields were determined. The results are shown in Table 1 below:

TABLE 1
Results of the experiments described above

| | | |
|---|---|---|
| Experiment | Yield | Conversion |

| | | | |
|---|---|---|---|
| Experiment 1 | Before | 47.5% | 52.7% |
| | After | 7.4% | 26.7% |
| Experiment 2 | Before | 24.5% | 45.8% |
| | After | 11% | 30.8% |
| Experiment 3 | Before | 38.6% | 54.7% |
| | After | 44% | 60.3% |
| Experiment 4 | Before | 44.9% | 61.7% |
| | After | 44.1% | 60.9% |

When purging with synthesis gas, the yields and conversions were found to return to a similarly high value after the idle time. Purging with nitrogen results in yields and conversions much lower than before the idle time.

1. A process for hydroformylating C2 to C8 olefins in a reaction zone in presence of a heterogenized catalyst system, the process comprising:
passing together a gaseous feed mixture containing the C2 to C8 olefins with synthesis gas in at least one reactor over a support arranged in the at least one reactor that is composed of a porous ceramic material on which a catalyst system, which comprises a metal from group 8 or group 9 of the periodic table of the elements, at least one organic phosphorus-containing ligand and a stabilizer, is present in a heterogenized form;
wherein the support is a monolith, or is present in a form of a powder, in a form of a granular material or in a form of shaped bodies and the support consists of a carbidic material, nitridic material, silicidic material or mixtures thereof,
wherein idle times occur during the process in which no gaseous feed mixture is passed through the at least one reactor, the at least one reactor being purged during idle times with synthesis gas or carbon monoxide.

2. The process according to claim 1, wherein the support does not have a washcoat applied, but is employed without washcoat.

3. The process according to claim 1, wherein the at least one organic phosphorus-containing ligand of the heterogenized catalyst system has the general formula (II) 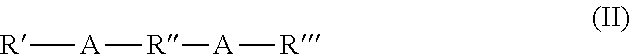
where R′, R″ and R′″ are each organic radicals, with proviso that R′ and R′″ are nonidentical, and both A are each a bridging —O—P(—O)₂ group, wherein two of the three oxygen atoms —O— are attached respectively to radical R′ and to radical R′″.

4. The process according to claim 1, wherein the stabilizer is an organic amine compound containing at least one 2,2,6,6-tetramethylpiperidine unit of formula (I):

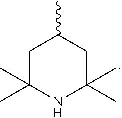

5. The process according to claim 1, wherein the nitridic material is selected from the group consisting of silicon nitride, boron nitride, aluminium nitride and mixtures thereof; the carbidic material is selected from the group consisting of silicon carbide, boron carbide, tungsten carbide or mixtures thereof; and the silicidic material is molybdenum silicide.

6. The process according to claim 5, wherein the support consists of a carbidic ceramic.

7. The process according to claim 6, wherein the support consists of silicon carbide.

8. The process according to claim 1, comprising:
carrying out the hydroformylation at a temperature in a range from 65 to 200° C.

9. The process according to claim 1, comprising:
hydroformulting at a pressure not greater than 35 bar.

10. The process according to claim 1, wherein the heterogenized catalyst system does not contain any ionic liquid.

11. The process according to claim 1, comprising:
employing C4 olefins in the hydroformylation process.

12. The process according to claim 1, wherein the employed metal from group 8 or 9 of the periodic table of the elements is rhodium.
